(19) United States
(12) Patent Application Publication
(10) Pub. No.: US 2026/0294328 A1
(43) Pub. Date: Oct. 1, 2026

(54) COMPOSITIONS AND METHOD FOR DIAGNOSING AND TREATING ALZHEIMER'S DISEASE

(71) Applicant: THE REGENTS OF THE UNIVERSITY OF CALIFORNIA, Oakland, CA (US)

(72) Inventor: Thomas JUE, Davis, CA (US)

(21) Appl. No.: 19/489,895

(22) PCT Filed: Jun. 5, 2024
(86) PCT No.: PCT/US2024/032564

§ 371 Date: Dec. 4, 2025

Related U.S. Application Data
(60) Provisional application No. 63/506,246, filed on Jun. 5, 2023.

(51) Int. Cl. A61B 5/00; A61B 5/055; A61K 31/19; A61K 45/06; A61K 49/10

(52) U.S. Cl. CPC A61B 5/4088; A61B 5/055; A61B 5/4064; A61K 31/19; A61K 45/06; A61K 49/10

(57) ABSTRACT

Disclosed herein are methods for diagnosing Alzheimer's disease in a subject that involves administering to the subject an imaging agent comprising hyperpolarized pyruvate having a nuclear magnetic resonance (NMR)-detectable nucleus; applying radiation to the brain of the subject, wherein the radiation has a frequency that excites electron spin transitions in the DNP agent at an intensity to polarize the NMR-detectable nucleus; detecting magnetic resonance signals in the brain of the subject from nuclear spin transitions in the NMR-detectable nucleus to produce a spectrum measuring bicarbonate (Bic) and lactate (Lac) metabolites; and calculating a Bic/Lac ratio from the measured metabolites, wherein a reduced Bic/Lac ratio compared to control is an indication that the subject has Alzheimer's disease.

### CROSS-REFERENCE TO RELATED APPLICATIONS

This application claims benefit of U.S. Provisional Application No. 63/506,246, filed Jun. 5, 2023, which is hereby incorporated herein by reference in its entirety.

### BACKGROUND OF THE INVENTION

Today, while the ultimate pathology of Alzheimer's disease is fairly well established, effective diagnostic methods and treatment modalities remain elusive because of the complex biological basis for the etiology and pathogenesis of the disease. Scientists and clinicians lack reliable diagnostic tests due to the absence of biologically specific screening techniques. Clinical diagnostic techniques for Alzheimer's disease currently rely on screening individuals displaying symptoms of dementia by excluding other possible causes such as depression, poor nutrition, other dementing conditions (e.g., Parkinson's disease with dementia), or drug interactions. These qualitative and unspecific methods often leave Alzheimer's disease misdiagnosed or unrecognized until later stages in the disease when treatments may be less effective. Early detection and treatment of Alzheimer's disease continues to be the best hope for successful treatment that may delay symptoms and extend a patient's quality of life.

### SUMMARY OF THE INVENTION

Disclosed herein is a method for diagnosing Alzheimer's disease in a subject that involves administering to the subject an imaging agent comprising hyperpolarized pyruvate having a nuclear magnetic resonance (NMR)-detectable nucleus; applying radiation to the brain of the subject, wherein the radiation has a frequency that excites electron spin transitions in the DNP agent at an intensity to polarize the NMR-detectable nucleus; detecting magnetic resonance signals in the brain of the subject from nuclear spin transitions in the NMR-detectable nucleus to produce a spectrum measuring bicarbonate (Bic) and lactate (Lac) metabolites; and calculating a Bic/Lac ratio from the measured metabolites, wherein a reduced Bic/Lac ratio compared to control is an indication that the subject has Alzheimer's disease.

In some embodiments, the method further involves administering to the subject an effective amount of a pyruvate dehydrogenase (PDH) stimulator, a pyruvate dehydrogenase kinase (PDK) inhibitor, or a combination thereof. For example, in some embodiments, the PDK inhibitor is dichloroacetate (DCA).

In some embodiments, the subject has been diagnosed with type 2 diabetes (T2D). In some embodiments, the method further involves repeating the method to map the Bic/Lac ratio over time.

In some embodiments, the NMR-detectable nucleus is a ¹³C in the C1 carbon of the pyruvate (¹³C-C1 pyruvate).

Also disclosed herein is a method for treating Alzheimer's disease in a subject that involves administering to the subject an effective amount of a pyruvate dehydrogenase (PDH) stimulator, a pyruvate dehydrogenase kinase (PDK) inhibitor, or a combination thereof. For example, the PDK inhibitor stimulator can be dichloroacetate (DCA).

The details of one or more embodiments of the invention are set forth in the accompanying drawings and the description below. Other features, objects, and advantages of the invention will be apparent from the description and drawings, and from the claims.

### BRIEF DESCRIPTION OF FIGURES

FIG. 1 shows two schemes for producing animal models having type 2 diabetes and Alzheimer's disease.

FIG. 2 shows colony founders with APP and PS1 transgenes and resulting glucose.

FIGS. 3A to 3C show amyloid burden in brain of T2D−/− rats at 6 months (FIG. 3A), T2D+/− rats at 6 months (FIG. 3B), and T2D+/− rats at 9 months (FIG. 3C). Propidium Iodide (PI) in red. F1-Fluoro-2,5-bis[(E)-3-carboxy-4-hydroxystyryl]benzene solution (FSB) in blue. Rats never spontaneously develop AD. T2D-AD^{+/-} shows amyloid burden at 6 mos. Tg344AD^{+/-} shows amyloid burden at 16 months.

FIGS. 4A to 4C show cognitive performance of T2D-AD^{+/-} and T2D-AD^{-/-} rats. FIG. 4A shows escape latency as a function of time for T2D-AD^{+/-} (square) and T2D-AD/(circle) rats.

FIGS. 4B and 4C show % use as a function of time for T2D-AD^{-/-} (FIG. 4B) and T2D-AD^{+/-} (FIG. 4C) rats.

FIG. 5 shows diabetic incidence (%) as a function of time for T2D-AD^{+/-} (dashed line) and T2D-AD/(solid line) rats. Diabetes incidence is glucose >350 mg/dL.

FIG. 6 shows non-fasting glucose levels (mg/dL) as a function of time for T2D-AD^{+/-} (dashed line) and T2D-AD/(solid line) rats.

FIG. 7 shows insulin levels (ng/mL) as a function of time for T2D-AD^{+/-} (dashed line) and T2D-AD^{-/-} (solid line) rats.

FIGS. 8A to 8C show Metabolite dynamics of Pyr, Lac and Bic signals in representative control (FIG. 8A), T2D-AD^{-/-} (FIG. 8B), and T2D-AD^{+/-} (FIG. 8C) animals after an injection of hyperpolarized ~100 mM [1-¹³C]Pyr solution.

FIG. 9 is a comparison of spectra from three representative animals highlights the increase in lactate and reduction of bicarbonate production in a T2D-AD^{-/-} animal compared to a control, which is further amplified in a T2D-AD^{+/-} animal.

FIG. 10 shows Bic/Lac expression in control, T2D-AD^{-/-}, and T2D-AD^{+/-}, Tg344-AD^{-/-}, and Tg344-AD^{+/-} rats.

FIG. 11 shows astrocyte neuron lactate shuttle (ANLS).

FIGS. 12A to 12C show metabolite dynamics of Pyr, Lac and Bic signals in representative control, T2D-AD^{-/-} and T2D-AD^{+/-} animals after an injection of hyperpolarized ~100 mM [1-¹³C]Pyr solution.

FIG. 13 contains a comparison of spectra from three representative animals highlights the increase in lactate and reduction of bicarbonate production in a T2D-AD−/− animal compared to a control, which is further amplified in a T2D-AD+/− animal.

FIG. 14 shows Bic/Lac in control, T2D-AD^{-/-}, T2D-AD^{+/-}, Tg344-AD^{-/-}, and Tg344-AD^{+/-} animals after an injection of hyperpolarized ~100 mM [1-¹³C]Pyr solution.

FIG. 15 illustrates the Astrocyte Neuron Lactate Shuttle (ANLS).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

### Definitions

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

### Dynamic Nuclear Polarization (DNP) NMR

Methods of using the DNP agents are provided. The methods can include MAS NMR methods, allowing for DNP enhancement in a site-specific manner. The methods can also include other standard NMR approaches including liquid NMR approaches and NMR approaches for medical applications. The methods can include dissolution DNP approaches used for MRI.

Methods of NMR measurement of an analyte comprising an NMR-detectable nucleus are provided. The NMR-detectable nucleus can be a half-integer spin nucleus, for example ¹H, ¹³C, ¹⁵N, or ¹⁹F. The analyte can be a protein or a nucleic acid. The methods can include interacting the analyte with a DNP agent provided herein. The DNP agent can react in a site-specific manner with the analyte, for example by forming a covalent bond with a thiol, amine, aldehyde, ketone, or other functional group on the analyte. The DNP agent can react in a site-specific manner with the analyte, for example by forming a non-covalent bond with a site in the analyte, such as a biotinylated site in a protein that interacts with avidin or an avidin derivative. The analyte can be associated with a membrane and the DNP agent can contain an amphiphilic group that non-covalently associates with the membrane.

The methods can include applying radiation having a frequency that excites electron spin transitions in the DNP agent in an intensity to polarize the NMR-detectable nucleus

The methods can include detecting a signal from nuclear spin transitions in the NMR-detectable nucleus. The signal can be greater than a second signal for the otherwise same NMR-detectable nucleus in the same analyte and taken under the same conditions except without the DNP agent in the sample. In some embodiments the signal is at least 2, 3, 4, 5, 10, 100, 500, or even 1,000 times as large as a second signal for the otherwise same NMR-detectable nucleus in the same analyte and taken under the same conditions except without the DNP agent in the sample.

The DNP procedure involves microwave irradiation of the electron paramagnetic resonance (EPR) spectrum of the DNP agent, and results in the transfer of the greater spin polarization of the electrons to the nuclei of surrounding nuclei at or near the site in the analyte. While the methods described herein are not limited to any specific magnetic field and the DNP procedure could be performed at low magnetic fields, the performance of dynamic nuclear polarization (DNP) experiments at the high magnetic fields used in contemporary NMR experiments (e.g., 5-20 T) is preferred.

A high frequency (140-600 GHz), low power (30 mW-3 W) solid state devices and high power (up to 5, 10, 20 W) microwave source can drive the DNP transitions associated with the second order electron-nuclear dipolar interactions in either continuous-wave (CW) or pulsed modes. To date the high power operation has been achieved by utilizing gyrotrons since they operate in the requisite frequency range and produce suitable microwave powers. The relaxation times of the electronic spin systems of radicals and bi- or oligo-radicals dictate that the methods be optimally performed at low temperatures (usually ≤90-120 K). When obtaining high resolution NMR spectra of solids, magic-angle spinning (MAS) is preferably incorporated into the experiment.

Hyperpolarization may be carried out by three possible mechanisms: (1) the Overhauser effect, (2) the solid effect and (3) thermal mixing effect (see A. Abragam and M. Goldman, Nuclear Magnetism: Order and Disorder, Oxford University Press, 1982). By hyperpolarization, it is meant that the sample is polarized to a level over that found at room temperature and 1 T, preferably polarized to a polarization degree in excess of 0.1%, more preferably 1%, even more preferably 10%. The Overhauser effect is the preferred method of the present invention though other methods are also anticipated. It is envisaged that, in the method according to the invention, the level of polarization achieved should be sufficient to allow the hyperpolarized solution of the carbon-13 enriched fullerenes and CNTs to achieve a diagnostically effective contrast enhancement in the sample to which it is subsequently administered in whatever form. In general, it is desirable to achieve a level of polarization which is at least a factor of 2 or more above the field in which MRI is performed, preferably a factor of 10 or more, particularly preferably 100 or more and especially preferably 1000 or more, e.g. 50000.

In another embodiment of the method according to the present invention, hyperpolarization of the MR imaging nuclei is effected by a DNP free radical source. In this embodiment, step (i) of the method comprises: (a) bringing an DNP free radical source and the carbon-13 enriched fullerene and CNTs into contact in a uniform magnetic field (the primary magnetic field Bo); (b) exposing said DNP free radical source to a first radiation of a frequency selected to excite electron spin transitions in said DNP free radical source; and (c) dissolving in a physiologically tolerable solvent said carbon-13 enriched fullerenes and CNTs. It is preferred that the DNP free radical source and carbon-13 enriched fullerene and CNTs are present as a composition during polarization.

### Metabolic Regulator

Disclosed herein are methods of treating Alzheimer's disease in a subject that involves administering to the subject a metabolic inhibitor in an effective amount to increase the ratio of Bic/Lac, i.e. to decrease lactate and/or or increase bicarbonate levels. Therefore, in some embodiments, the metabolic regulator is a pyruvate dehydrogenase (PDH) stimulator, a pyruvate dehydrogenase kinase (PDK) inhibitor, or a combination thereof.

Pyruvate dehydrogenase kinase family members (PDK1, PDK2, PDK3, PDK4) are serine kinases that catalyze phosphorylation of the E1a subunit of the pyruvate dehydrogenase complex (PDC). Pyruvate dehydrogenase kinase is activated by ATP, NADH and acetyl-CoA. It is inhibited by ADP, NAD+, CoA-SH and pyruvate. Biochemicals that inhibit PDK may be used to direct hematopoietic lineage biasing and to generate definitive hematopoietic cells. For example, inhibitors of Pyruvate dehydrogenase kinases (PDK) include Leelamine HCl, a weak CB1 receptor agonist and PDK inhibitor; Quercetin Dihydrate, a natural flavonoid antiproliferative kinase inhibitor; Sodium dichloroacetate, an inhibitor of mitochondrial pyruvate dehydrogenase kinase; SB 203580 (hydrochloride), a MAPK inhibitor; Dichloroacetic acid, a mitochondrial PDK (pyruvate dehydrogenase kinase) inhibitor; PDK1/Akt/Flt Dual Pathway Inhibitor, which is a cell-permeable compound that selectively induces apoptosis; BX 795, an inhibitor of PDK1, TBK1, and IKK&epsilon; SB 203580; a pyridinyl imidazole and specific inhibitor that suppresses p38 mediated activation of MK2; KT 5720, a potent, specific and cell-permeable inhibitor of PKA; BX-912, a potent and selective PDK-1 inhibitor that induces apoptosis; GSK 2334470, a potent and selective PDK1 inhibitor that subsequently induces apoptotic cell death; and OSU 03012, a PDK1 inhibitor and inducer of caspase and p53-independent apoptosis.

Pyruvate dehydrogenase (PDH) is the first component enzyme of pyruvate dehydrogenase complex (PDC). The pyruvate dehydrogenase complex contributes to transforming pyruvate into acetyl-CoA by a process called pyruvate decarboxylation (Swanson Conversion). Acetyl-CoA may then be used in the citric acid cycle to carry out cellular respiration. Thus, pyruvate dehydrogenase links the glycolysis metabolic pathway to the citric acid cycle and releasing energy via NADH. Pyruvate dehydrogenase may be allosterically activated by fructose-1,6-bisphosphate and is inhibited by NADH and acetyl-CoA. Phosphorylation of PDH is mediated by pyruvate dehydrogenase kinase. Metabolic regulators may be used that activate Pyruvate Dehydrogenase complexes (PDH).

In some examples, metabolic regulators may be used to increase uptake of pyruvate into mitochondria. Transport of pyruvate across the outer mitochondrial membrane (OMM) is accomplished via large non-selective channels, such as voltage-dependent anion channels/porin, which enable passive diffusion (Benz R. Biochim Biophys Acta. 1994; 1197:167-196). Voltage-Dependent Anion Channel (VDAC) is the most abundant protein in the OMM and serves as the main pathway for metabolite/ion transport between the cytosol and the intermembrane space (IMS) of mitochondria. Deficiencies in these channels have been suggested to block pyruvate metabolism (Huizing M. et al. Pediatr Res. 1996; 39:760-765). Inhibitors of voltage-dependent anion channels/porin may be used to inhibit uptake of pyruvate. VDAC phosphorylation by protein kinases, GSK3B, PKA, and protein kinase C epsilon (PKCε), blocks or inhibits association of VDAC with other proteins, such as Bax and tBid, and also regulates VDAC opening. PKA-dependent VDAC phosphorylation and GSK3β-mediated VDAC2 phosphorylation increase VDAC conductance.

The movement of metabolites, such as pyruvate, through the inner mitochondrial membrane (IMM) may be more restrictive than across the OMM, however. Many metabolites have specific mitochondrial inner membrane transporters that have been identified and studied (Palmieri F. et al. Biochim Biophys Acta. 1996; 1275:127-132).

Metabolic regulators may be used that accelerate conversion of pyruvate to acetyl coenzyme A (Ac-CoA). Dichloroacetate (DCA) promotes pyruvate entry into the Krebs cycle by inhibiting pyruvate dehydrogenase (PDH) kinase and thereby maintaining PDH in the active dephosphorylated state. In instances where the metabolic regulator is dichloroacetate (DCA), the concentration of dichloroacetate in a culture media for the source cells may be at least about 30 UM and can vary from 10 UM to 100 UM, including concentrations of about: 10 UM, 20 UM, 30 μM, 40 UM, 50 M, 60 UM, 70 UM, 80 UM, 90 UM and 100 μM.

### EXAMPLES

### Example 1: Rat Model of Type 2 Diabetes (T2D) with Alzheimer's Disease (AD)

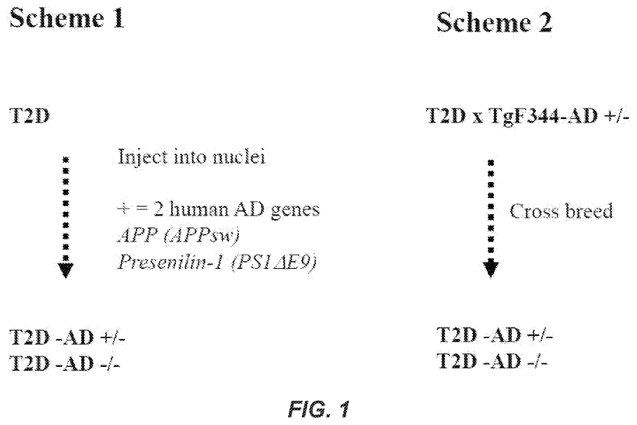

FIG. 1 shows two schemes for producing animal models having type 2 diabetes and Alzheimer's disease.

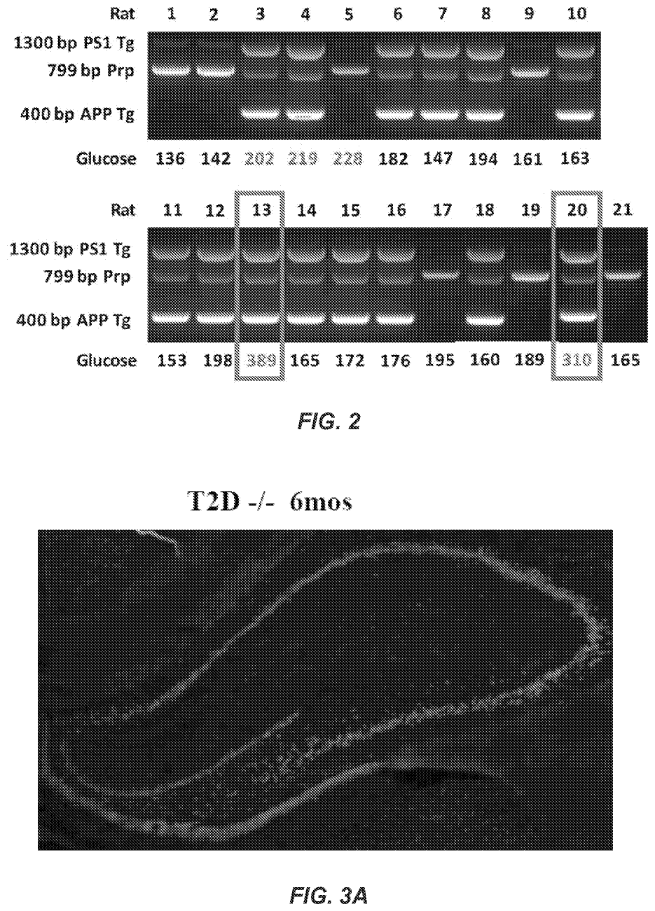

FIG. 2 shows colony founders with APP and PS1 transgenes and resulting glucose.

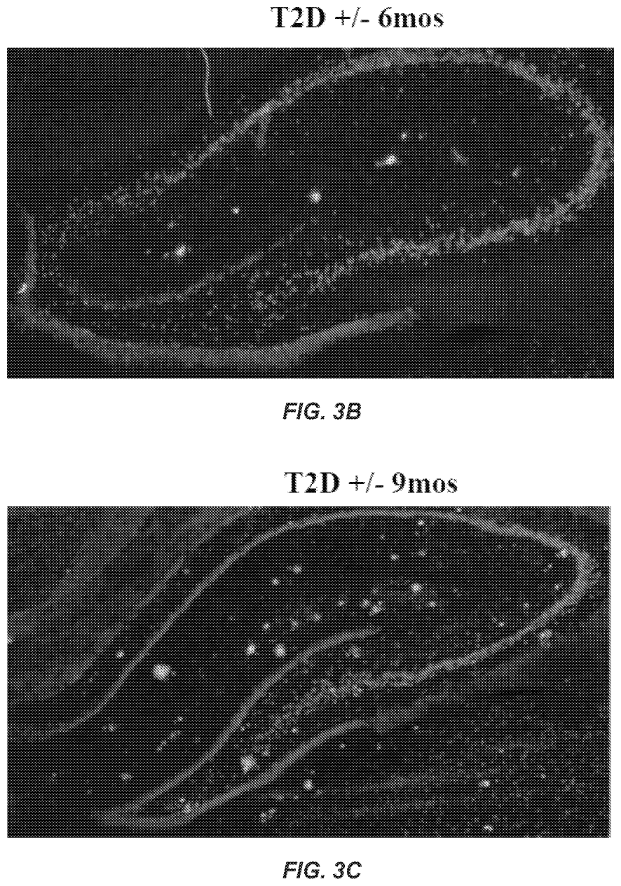

FIGS. 3A to 3C show amyloid burden in brain of T2D−/− rats at 6 months (FIG. 3A), T2D+/− rats at 6 months (FIG. 3B), and T2D+/− rats at 9 months (FIG. 3C). Propidium Iodide (PI) in red. F1-Fluoro-2,5-bis[(E)-3-carboxy-4-hydroxystyryl]benzene solution (FSB) in blue. Rats never spontaneously develop AD. T2D-AD^{+/-} shows amyloid burden at 6 mos. Tg344AD^{+/-} shows amyloid burden at 16 months.

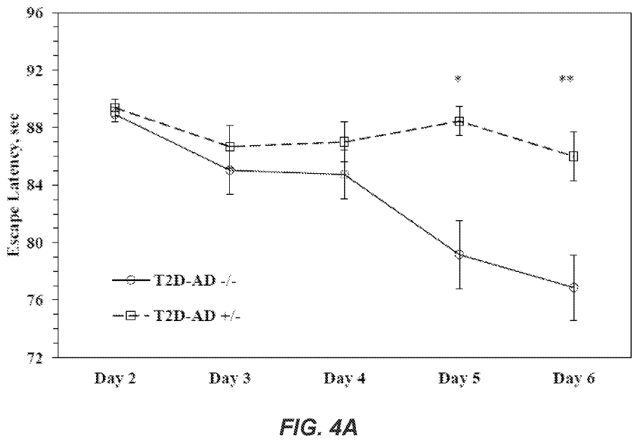

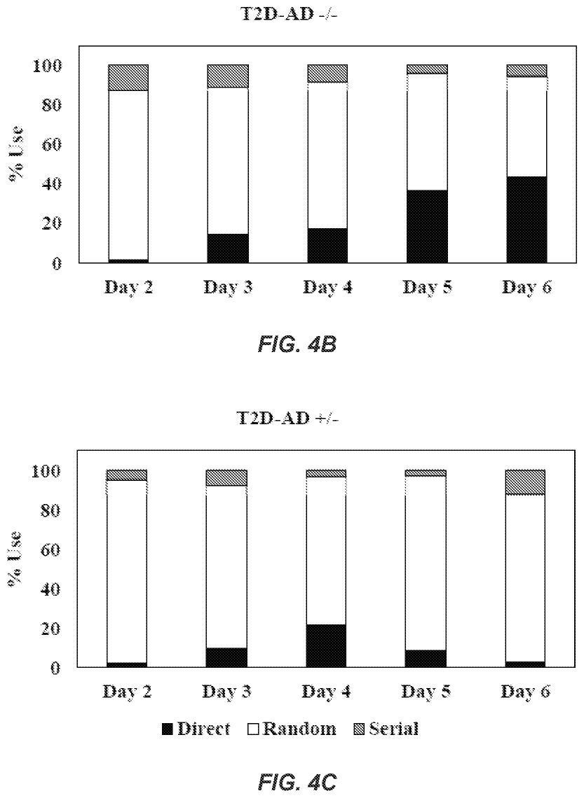

FIGS. 4A to 4C show cognitive performance of T2D-AD^{+/-} and T2D-AD^{-/-} rats. FIG. 4A shows escape latency as a function of time for T2D-AD^{+/-} (square) and T2D-AD^{-/-} (circle) rats. FIGS. 4B and 4C show % use as a function of time for T2D-AD^{-/-} (FIG. 4B) and T2D-AD^{+/-} (FIG. 4C) rats.

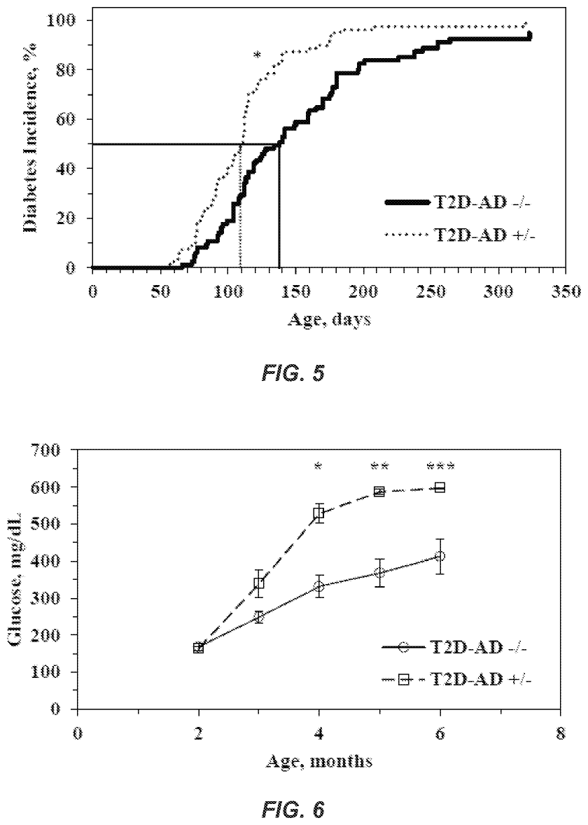

FIG. 5 shows diabetic incidence (%) as a function of time for T2D-AD^{+/-} (dashed line) and T2D-AD^{-/-} (solid line) rats. Diabetes incidence is glucose >350 mg/dL.

FIG. 6 shows non-fasting glucose levels (mg/dL) as a function of time for T2D-AD^{+/-} (dashed line) and T2D-AD^{-/-} (solid line) rats.

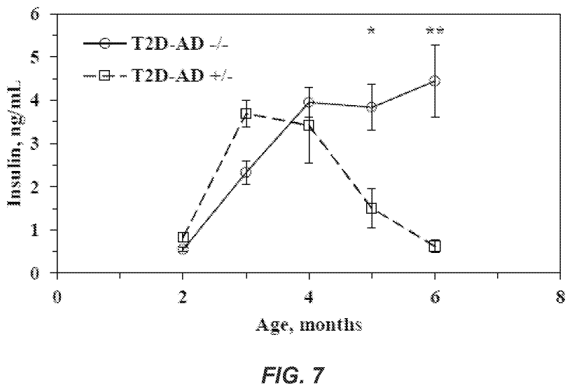

FIG. 7 shows insulin levels (ng/ml) as a function of time for T2D-AD^{+/-} (dashed line) and T2D-AD^{-/-} (solid line) rats.

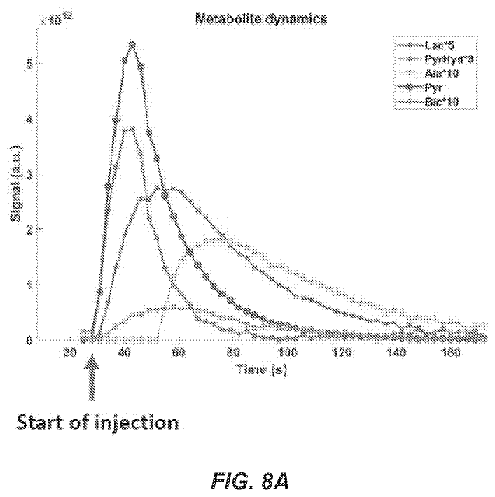

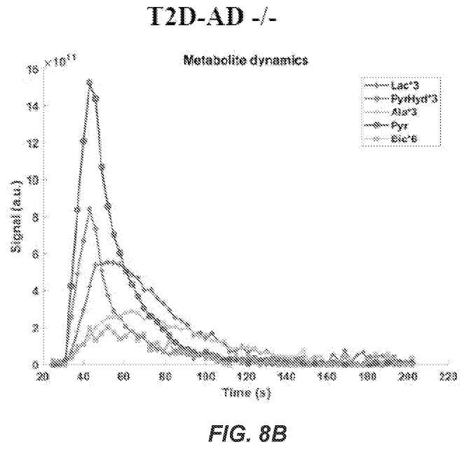

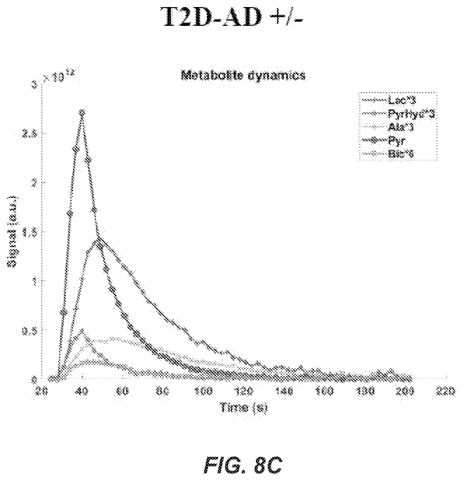

FIGS. 8A to 8C show Metabolite dynamics of Pyr, Lac and Bic signals in representative control (FIG. 8A), T2D-AD/(FIG. 8B), and T2D-AD^{+/-} (FIG. 8C) animals after an injection of hyperpolarized ~100 mM [1-¹³C]Pyr solution.

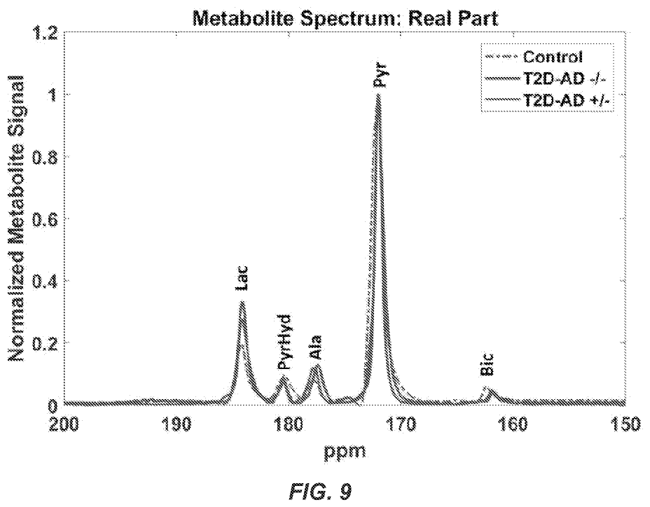

FIG. 9 is a comparison of spectra from three representative animals highlights the increase in lactate and reduction of bicarbonate production in a T2D-AD^{-/-} animal compared to a control, which is further amplified in a T2D-AD^{+/-} animal.

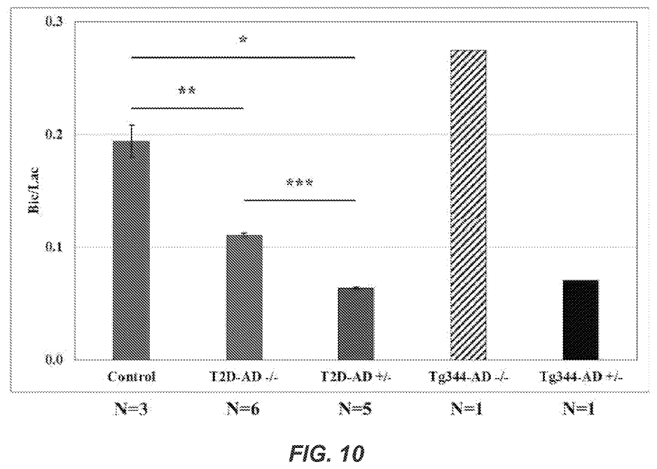

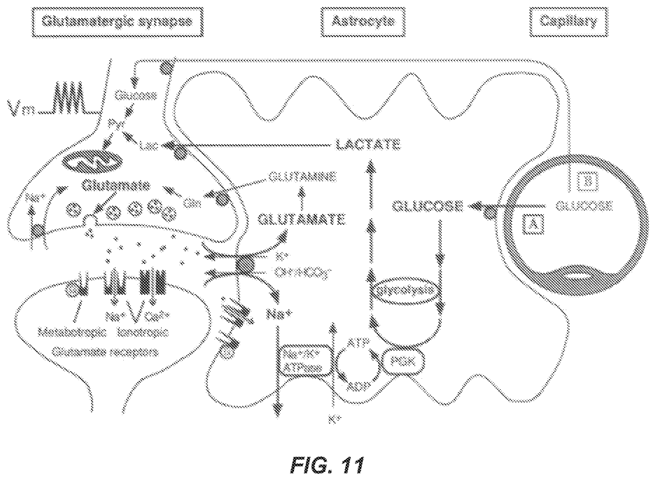

FIG. 10 shows Bic/Lac expression in control, T2D-AD^{-/-}, and T2D-AD^{+/-}, Tg344-AD^{-/-}, and Tg344-AD^{+/-} rats.

TABLE 1

| | F-statistics | Df | N | P |
|---|---|---|---|---|
| Ctrl vs. T2D-AD^{+/-} | 140 | 6 | 7 | 7.6 × 10⁻⁵ |
| Ctrl vs. T2D-AD^{-/-} | 50 | 7 | 8 | 4.1 × 10⁻⁴ |
| T2D-AD^{-/-} vs. T2D-AD^{+/-} | 105 | 10 | 11 | 3.0 × 10⁻⁶ |

TABLE 2

| | Average ± Std. Error |
|---|---|
| Control | 0.194 ± 0.014 |
| T2D-AD^{-/-} | 0.111 ± 0.002 |
| T2D-AD^{+/-} | 0.064 ± 0.001 |
| Tg344-AD^{-/-} | 0.275 |
| Tg344-AD^{+/-} | 0.071 |

FIG. 11 shows astrocyte neuron lactate shuttle (ANLS).

Therefore, this example provides an animal model with T2D and AD transgenes. The Tg344AD and T2D-AD transgenes have the same transgene intercalation site.

In these rats, amyloid burden appears early (6 mos). In addition, there is a cognitive performance: T2D-AD+/− poorer than T2D−/−. PDH activity decreases with T2D and further with T2D-AD. DCA appears to restore PDH activity. And, finally, AD and T2D have reciprocal effects.

### Example 2: Dynamic Nuclear Polarization (DNP)

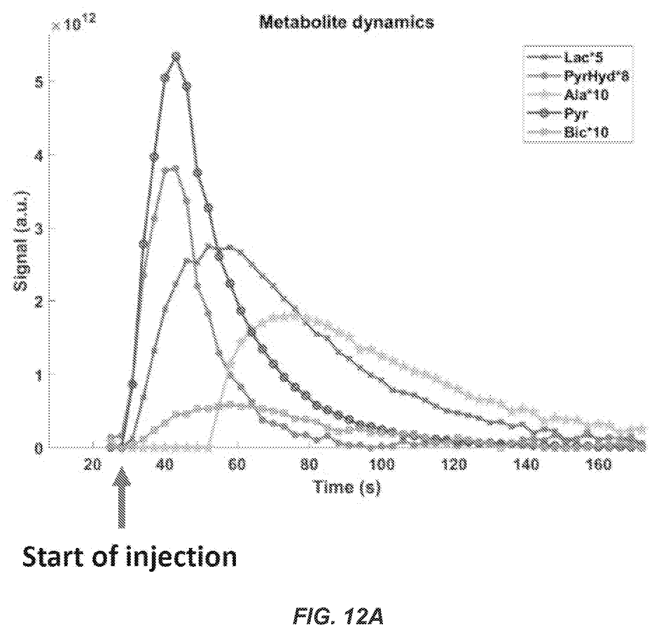

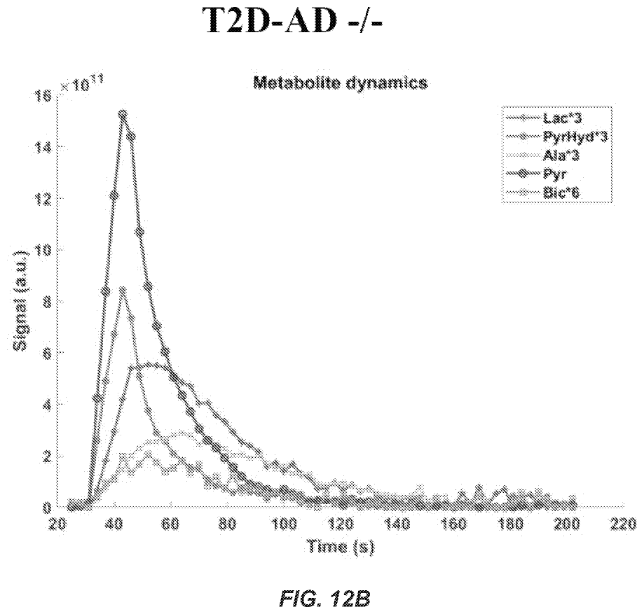

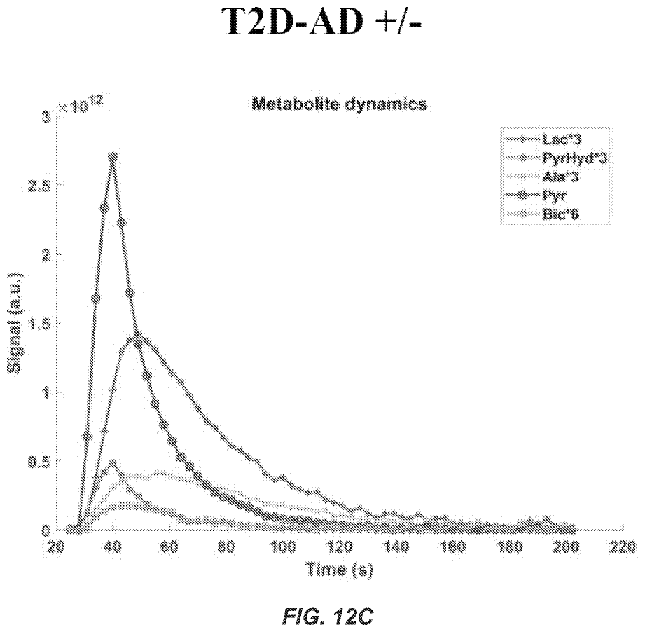

FIGS. 12A to 12C show metabolite dynamics of Pyr, Lac and Bic signals in representative control, T2D-AD^{-/-} and T2D-AD^{+/-} animals after an injection of hyperpolarized ~100 mM [1-¹³C]Pyr solution.

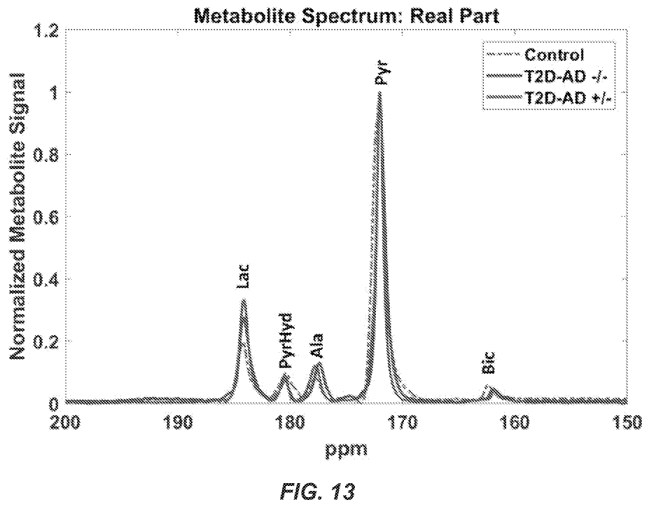

FIG. 13 contains a comparison of spectra from three representative animals highlights the increase in lactate and reduction of bicarbonate production in a T2D-AD^{-/-} animal compared to a control, which is further amplified in a T2D-AD+/− animal.

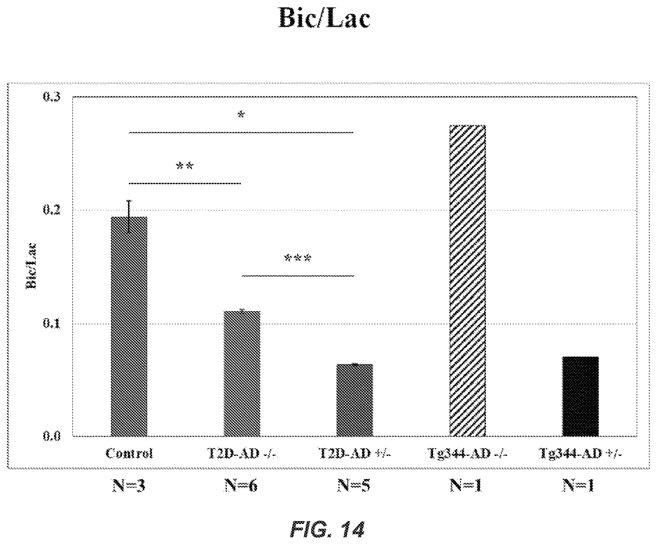

FIG. 14 shows Bic/Lac in control, T2D-AD^{-/-}, T2D-AD^{+/-}, Tg344-AD^{-/-}, and Tg344-AD^{+/-} animals after an injection of hyperpolarized ~100 mM [1-¹³C]Pyr solution.

TABLE 3

| | F-statistic | Df | N | p |
|---|---|---|---|---|
| Ctrl vs. T2d-AD^{+/-} | 140 | 6 | 7 | 7.6 × 10⁻⁵ |
| Ctrl vs. T2d-AD^{-/-} | 50 | 7 | 8 | 4.1 × 10⁻⁴ |
| T2d-AD^{-/-} vs. T2d-AD^{+/-} | 105 | 10 | 11 | 3.0 × 10⁻⁶ |

TABLE 4

| | Average ± Std. Error |
|---|---|
| Control | 0.194 ± 0.014 |
| T2d-AD^{-/-} | 0.111 ± 0.002 |
| T2d-AD^{+/-} | 0.064 ± 0.001 |
| Tg344-AD^{-/-} | 0.275 |
| Tg344-AD^{+/-} | 0.071 |

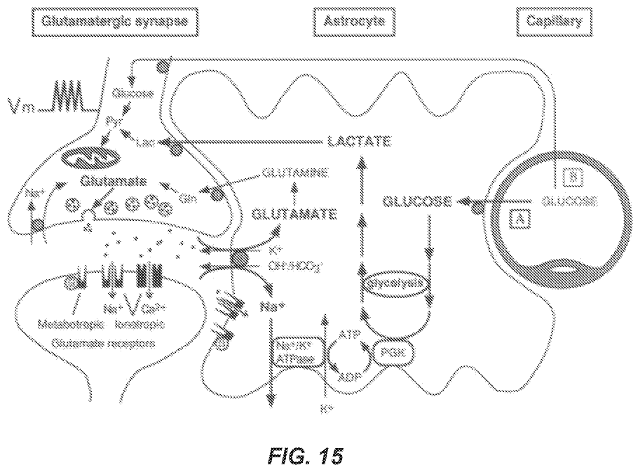

FIG. 15 illustrates the Astrocyte Neuron Lactate Shuttle (ANLS). Oxidative metabolism (neuron) decreases in T2D-AD. Without wishing to be bound by theory, it is possible that T2D-AD alters the ANLS.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for diagnosing Alzheimer's disease in a subject, comprising
(a) administering to the subject an imaging agent comprising hyperpolarized pyruvate having a nuclear magnetic resonance (NMR)-detectable nucleus,
(b) applying radiation to the brain of the subject, wherein the radiation has a frequency that excites electron spin transitions in the DNP agent at an intensity to polarize the NMR-detectable nucleus;
(c) detecting magnetic resonance signals in the brain of the subject from nuclear spin transitions in the NMR-detectable nucleus to produce a spectrum measuring bicarbonate (Bic) and lactate (Lac) metabolites; and
(d) calculating a Bic/Lac ratio from the measured metabolites,
wherein a reduced Bic/Lac ratio compared to control is an indication that the subject has Alzheimer's disease.

2. The method of claim 1, further comprising administering to the subject an effective amount of a pyruvate dehydrogenase (PDH) stimulator, a pyruvate dehydrogenase kinase (PDK) inhibitor, or a combination thereof.

3. The method of claim 2, wherein the PDK inhibitor comprises dichloroacetate (DCA).

4. The method of claim 1, wherein the subject has been diagnosed with type 2 diabetes (T2D).

5. The method of claim 1, further comprising repeating steps (a) to (d) to map the Bic/Lac ratio over time.

6. The method of claim 1, wherein the NMR-detectable nucleus comprises 13C in the C1 carbon of the pyruvate (13C-C1 pyruvate).

7. The method for treating Alzheimer's disease in a subject, comprising administering to the subject an effective amount of a pyruvate dehydrogenase (PDH) stimulator, a pyruvate dehydrogenase kinase (PDK) inhibitor, or a combination thereof.

8. The method of claim 6, wherein the PDK inhibitor stimulator comprises dichloroacetate (DCA).
